(19) United States
(12) Patent Application Publication
(10) Pub. No.: US 2019/0247630 A1
(43) Pub. Date: Aug. 15, 2019

(54) BALLOON CATHETER FOR CONTRAST AGENT FILTRATION AND REMOVAL

(71) Applicant: Teleflex Innovations S.à.r.l., Luxembourg City (LU)

(72) Inventor: Joshua Brenizer, Maple Grove, MN (US)

(21) Appl. No.: 16/264,790

(22) Filed: Feb. 1, 2019

Related U.S. Application Data
(60) Provisional application No. 62/630,468, filed on Feb. 14, 2018.

(51) Int. Cl. A61M 25/10; A61M 5/00; A61M 25/00

(52) U.S. Cl. CPC A61M 25/10; A61M 5/007; A61M 25/0075; A61M 2202/0413; A61M 2025/1052; A61M 2210/125; A61M 2025/0037

(57) ABSTRACT

Balloon catheters for, and related methods of, preventing contrast-associated nephropathy are disclosed. A method for preventing contrast-associated nephropathy can include inserting a catheter into a patient's coronary sinus and blocking blood flow from the coronary sinus into the patient's right atrium. The blood may contain a contrast agent. The method can further include draining the blood from the coronary sinus through a first lumen defined by the catheter while the blood flow is blocked, filtering the contrast agent from the blood which passes through the catheter, and returning contrast-free or contrast-reduced blood to the right atrium through a second lumen defined by the catheter, which terminates in a port proximal to the coronary sinus. Another method can include selectively draining blood from the coronary sinus into the right atrium through a first lumen defined by a catheter that includes a switchable valve while the blood flow is blocked.

### CLAIM OF PRIORITY

This non-provisional patent document claims the benefit of priority under 35 U.S.C. § 119(e) to U.S. Provisional Patent Application Ser. No. 62/630,468, entitled “BALLOON CATHETER FOR CONTRAST AGENT FILTRATION AND REMOVAL” and filed on Feb. 14, 2018, which is herein incorporated by reference in its entirety.

### TECHNICAL FIELD

This patent document relates to medical devices. More particularly, but not by way of limitation, this patent document relates to balloon catheters.

### BACKGROUND

During a percutaneous coronary interventional (PCI) procedure, a solution containing dye or contrast agent is often injected into a main coronary artery through a guide or diagnostic catheter. The contrast agent can be fluorescent, enabling the coronary artery and its branches to be visualized, thereby allowing for identification of a stenosis. The use of a contrast agent, however, creates a certain amount of risk, especially in patients with kidney disease. For instance, patients commonly require revascularization of more than a single vessel, but when there is danger of contrast-induced renal failure, the vessels must be treated on separate visits, which is costly and inconvenient but necessary to avoid kidney failure. General dialysis is not an adequate solution because the concentration of contrast agent in the blood is low at dialysis locations, e.g., at a wrist or forearm, and flow rates of dialysis procedures are too high for patients undergoing PCI procedures.

### Overview

The present inventor recognizes that there is a need to reduce the incidence of contrast-associated kidney failure and other issues caused by PCI procedures that employ contrast agents. Based on the understanding that renal issues induced by contrast agents can be prevented if the contrast agents do not travel to the kidneys, the inventor conceived and developed new devices and methods for collecting and filtering contrast-heavy blood, or selectively collecting and discarding contrast-heavy blood altogether. A balloon catheter defining a distal port and either a proximal port or proximal valve can be used to filter contrast agent from blood collected in the coronary sinus or discard the contrast-heavy blood in a controlled manner, respectively, such that only contrast-free or contrast-reduced blood enters the right atrium during a PCI procedure that employs contrast material. Depending on the number of ports included in the catheter, the number of lumens defined within the catheter can also vary. Two or more lumens can each be independently coupled with an external device configured to inflate the catheter balloon, filter contrast-heavy blood, or receive discarded contrast-heavy blood.

These and other embodiments and features of the present balloon catheters and related methods will be set forth, at least in part, in the following Detailed Description. This Overview is intended to provide non-limiting embodiments of the present subject matter—it is not intended to provide an exclusive or exhaustive explanation of the disclosed embodiments. The Detailed Description below is included to provide further information about the present balloon catheters and methods.

### BRIEF DESCRIPTION OF THE DRAWINGS

In the drawings, like numerals can be used to describe similar features and components throughout the several views. The drawings illustrate generally, by way of example, but not by way of limitation, various embodiments discussed in this patent document.

FIG. 1 illustrates a schematic view of a balloon catheter and an anatomical view of a heart, where the balloon catheter is positioned to perform a PCI.

FIG. 2 illustrates a schematic view of a balloon catheter inserted into the coronary sinus of a heart, where the balloon catheter comprises proximal and distal ports positioned on opposite sides of the balloon.

FIG. 3 illustrates a schematic magnified view of a portion of the balloon catheter shown in FIG. 2.

FIG. 4 illustrates a schematic view of a balloon catheter inserted into the coronary sinus of a heart, where the balloon catheter comprises a distal port and a proximal valve positioned on opposite sides of the balloon.

FIG. 5 illustrates a schematic magnified view of the balloon catheter of FIG. 4 in a closed configuration.

FIG. 6 illustrates a schematic magnified view of the balloon catheter of FIG. 4 in an open configuration.

The drawings are not necessarily to scale. Certain features and components may be shown exaggerated in scale or in schematic form, and some details may not be shown in the interest of clarity and conciseness.

### DETAILED DESCRIPTION

This patent document discloses multi-lumen balloon catheters configured to prevent contrast-heavy blood injected into a patient's coronary artery from traveling to the patient's kidneys. Contrast agents utilized to assess blood flow and occlusions during PCI procedures may be provided as a solution, the concentration of which may be the highest in a patient's coronary sinus, which is a collection of veins joined together to form a large vessel that collects blood from the heart muscle. From the coronary sinus, contrast-heavy blood flows into the right atrium and eventually to the kidneys. By blocking blood flow from the coronary sinus to the right atrium and removing contrast-heavy blood from the sinus, either temporarily or permanently, the balloon catheters of the present invention can reduce or eliminate the likelihood of contrast-associated nephropathy caused by PCI procedures.

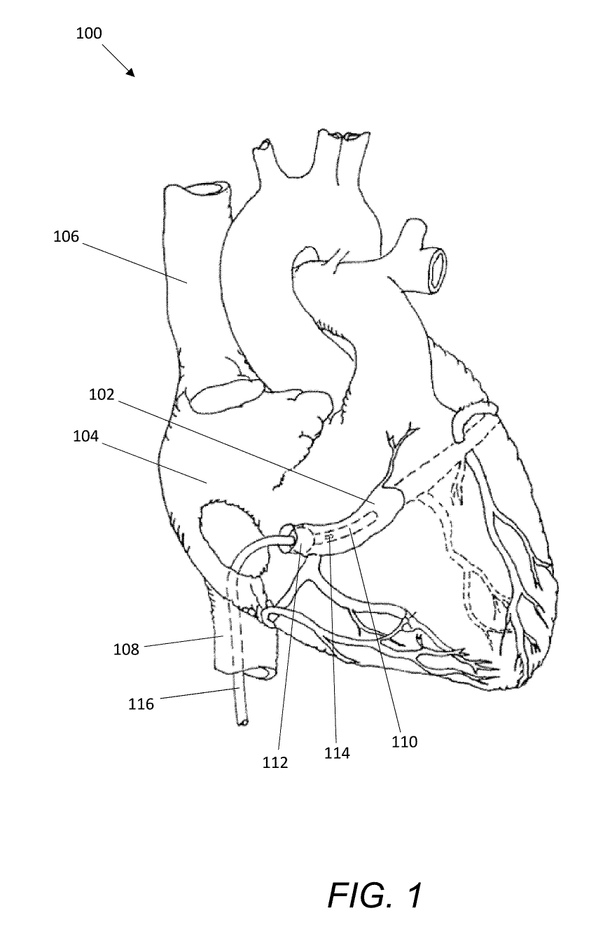

FIG. 1 provides anatomical context for the devices and methods described herein, showing the heart 100, coronary sinus 102, right atrium 104, superior vena cava 106, and inferior vena cava 108. A balloon catheter 110 is shown inserted into the coronary sinus 102 such that the balloon 112 is positioned to block contrast-heavy blood from flowing proximally out of the coronary sinus 102 and into the right atrium 104, and a distal port 114 is positioned to receive the blood within the sinus. A proximal portion 116 of the catheter (relative to the balloon) can extend through the inferior vena cava 106 toward the catheter entry point, e.g., the groin region. The balloon 112 can be inflated and deflated responsive to an external inflation device. The catheter 110 can be inserted into the position shown in FIG. 1 according to medical techniques known in the art.

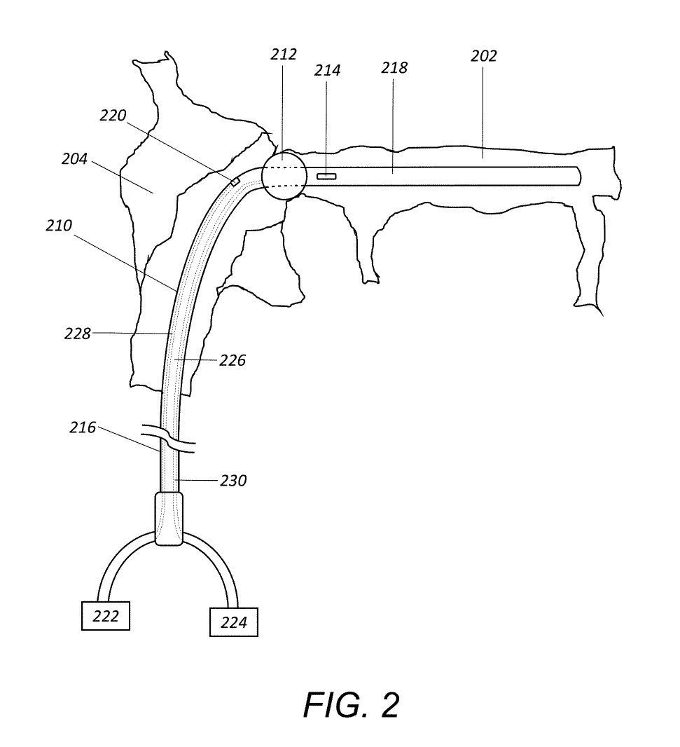

FIG. 2 shows a schematic view of a balloon catheter 210 inserted into a heart according to the present invention. In the embodiment shown, only a single catheter, and thus a single access point, is sufficient to remove contrast-heavy blood from the coronary sinus and return contrast-free or contrast-reduced blood to the right atrium. The balloon catheter 210 can be passed over a guidewire such that a distal portion 218 of the catheter, which defines a distal port 214, extends into the coronary sinus 202. The balloon 212 of the balloon catheter 210 is positioned proximal to the distal port 214, between the coronary sinus 202 and the right atrium 204, preventing blood flow therebetween when inflated. The balloon catheter 210 also defines a proximal port 220, which is configured to direct the flow of contrast-free or contrast-reduced blood back into the right atrium 204. The catheter 210 is coupled, at a proximal portion 216, to a filtration device 222 and an inflation device 224. The catheter 210 can be coupled with additional or alternative devices in other examples, such as a pressurization device configured to monitor the internal pressure of the coronary sinus, or a contrast agent injection device, among others. In some examples, the catheter 210 can include one or more anchoring, positioning, and/or stabilizing components configured to maintain the position of the balloon catheter 210 illustrated in FIG. 2. In some examples, the catheter 210 can include a non-inflatable, expandable member in lieu of the balloon 212. The catheter 210 may also include one or more electrophysiology sensing devices, e.g., electrodes, configured to perform various diagnostic functions within the ostium of the coronary sinus.

As further shown, the catheter 210 may generally comprise an elongate tube member, which can define one or more preformed curvatures configured specifically for insertion into the coronary sinus ostium 202. In various embodiments, the catheter 210 may define a longitudinal curve positioned proximal to the balloon 212. Such a preformed curve may define an arc of about 30° to about 50° over a length of about 7.0 to 11.0 cm. In some examples, the catheter 210 may define one or more additional curved portions, e.g., a distal hook of about 50° to 70° spanning a length of about 0.5 to 2.0 cm. The rigidity of the elongate tube member may vary along its length, such that certain portions are more flexible, and thus amenable to manipulation, than other portions, which may comprise more rigid materials and/or reinforcement components. The catheter 210 may comprise a soft distal tip portion, and in some examples, the catheter 210 may transition from less pliable to more pliable moving distally along its length. The proximal portion 216 can be the straightest and most rigid portion of the catheter in various embodiments. The length of the elongate tube defined by the catheter 210 can range from about 15 cm to about 130 cm. The cross-sectional diameter of the catheter 210 may also vary, ranging from about 3F, 4F, 5F, 6F, 7F, 8F or larger. Methods of forming the catheter 210 may involve extruding one or more components.

In operation, contrast-heavy blood collected in the coronary sinus 202 flows into the distal portion 218 of the catheter 210 via distal port 214, which defines the distally terminal portion of a first lumen 226. The contrast-heavy blood is moved proximally through the first lumen 226, traveling from the coronary sinus 202 to the filtration device 222, which separates the contrast agent from the blood. After filtration, the contrast-free or contrast-reduced blood is returned to the right atrium 204 through a second lumen 228, which terminates at proximal port 220, positioned within the right atrium 204. Prior to its recirculation, the contrast-free or contrast-reduced blood may be mixed with an amount of water and/or saline to maintain a constant blood volume within the patient.

A third lumen 230 connects the inflation device 224 to the balloon 212, such that inflation device 224 can inflate the balloon prior to injection of the contrast agent, and deflate the balloon after removal of the contrast-heavy blood. In various embodiments, the balloon 212 can remain inflated even when no contrast agent is being injected. Air and/or liquid inflation materials may be used to expand the balloon 212.

As further shown, the second and third lumens 228, 230 can define distinct channels within the catheter 210, while the first lumen 226 can comprise the remainder of the interior volume of the catheter. In some examples, the second and third lumens 228, 230 can be integrated into the external walls of the catheter 210. In additional examples, the first lumen 226 can also comprise a distinctly-defined tube within the interior volume of the catheter 210. Regardless of the specific arrangement, the lumens 226, 228, 230 may define independent channels within the catheter 210. The proportion of available channel volume occupied by each lumen may vary. In some examples, the first lumen 226 occupies the greatest proportion of total volume within the catheter 210, while in additional examples, the first and second lumens 226, 228 each occupy an equal proportion of the total channel volume, such that the rate contrast-heavy blood removal approximately matches the rate of contrast-free or contrast-reduced blood replenishment. The configuration of the balloon 212 can also vary. The balloon 212 is spherical in the example shown, but the balloon may also be conical, square, elongate, tapered, stepped, offset, rectangular, and/or combinations thereof.

Filtration device 222 may be configured to perform multiple varieties of filtration. For example, filtration device 222 may include a centrifuge configured to separate contrast agent from liquid. In some examples, filtration device 222 may be configured to perform dialysis or continuous veno-venous hemodiafiltration (CVVHD). The balloon catheter 210 can be coupled with any filtration means known to one skilled in the art. Various contrast agents may also be used, and may impact the type of filtration performed. For example, contrast agents may be radiopaque and can be water soluble or water insoluble. Particular contrast agents employed may include metrizamide, iopamidol, iothalamate sodium, iodamide sodium, meglumine, and metals and metal oxides such as titanium, gold, silver, stainless steel, oxides thereof, aluminum oxide, and/or zirconium oxide.

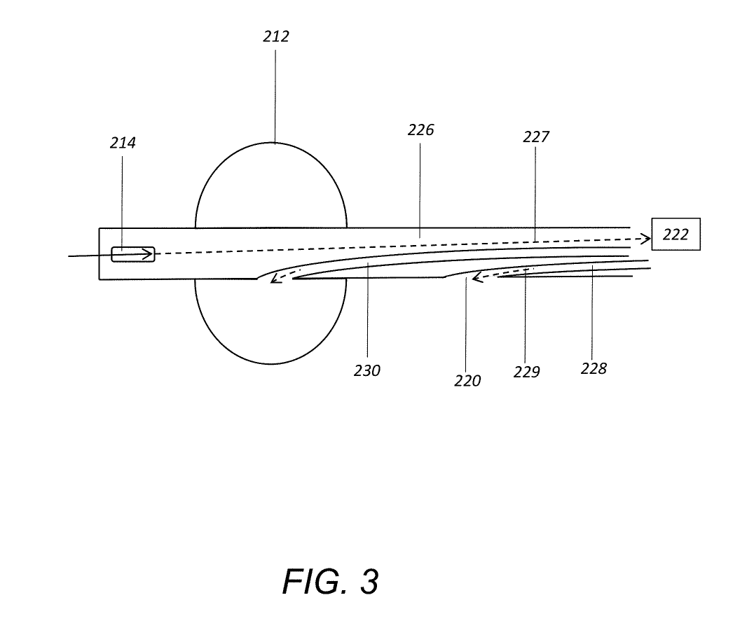

FIG. 3 shows a magnified schematic view of a portion of the balloon catheter 210 shown in FIG. 2, arranged in an opposite orientation, such that the distal port 214 is shown on the left. As shown, contrast-heavy blood 227 enters the distal port 214 and flows proximally through the first lumen 226 toward a filtration device 222. After filtration, contrast-free or contrast-reduced blood 229 is guided through the second lumen 228 to distal port 220, where the blood exits the catheter 210 and re-enters the right atrium for eventual even circulation to the kidneys. The third lumen 230, which may be referred to as the inflation lumen, connects the balloon 212 with an external inflation device.

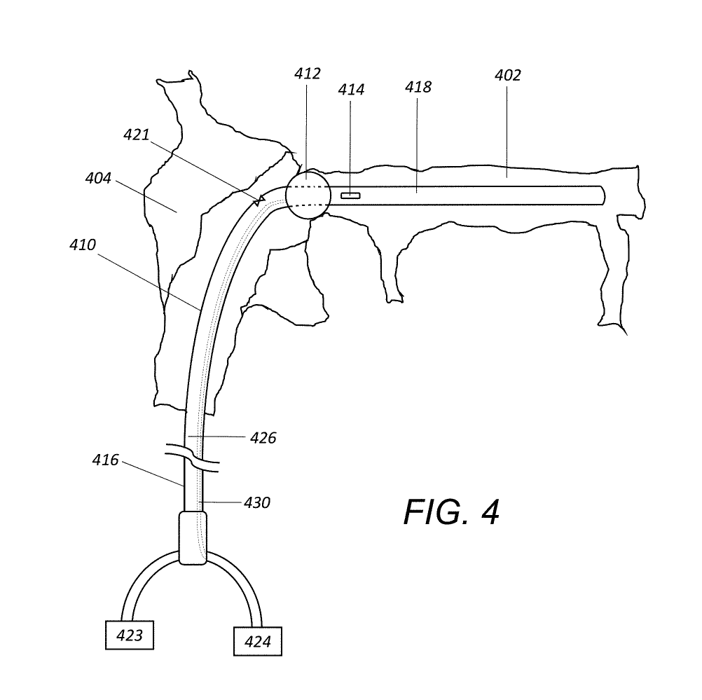

FIG. 4 shows a schematic view of a balloon catheter 410 inserted into a heart according to another embodiment of the present invention. According to this embodiment, only a single catheter, and thus a single access point, is again sufficient to remove contrast-heavy blood from the coronary sinus and return contrast-free or contrast-reduced blood to the right atrium. Like balloon catheter 210, the balloon catheter 410 shown in FIG. 4 can be passed over a guidewire such that a distal portion 418 of the catheter, which defines a distal port 414, extends into the coronary sinus 402. The balloon 412 of the balloon catheter 410 is positioned proximal to the distal port 414, between the coronary sinus 402 and the right atrium 404, preventing blood flow therebetween when inflated. The balloon catheter 410 also defines a proximal valve 421, which is configured to selectively allow blood flow back into the right atrium 404. The catheter 410 is coupled, at a proximal portion 416, to a drainage device 423 and an inflation device 424. The inflation device 424 is coupled to the balloon 412 via an inflation lumen 430. The length and curvature of the catheter 410 may be generally the same as the configuration of the catheter 210 shown and described in FIG. 2. In addition, the proportion of the interior volume defined by each independent channel of the elongate tubular portion of the catheter 410 may vary. In some examples, the first lumen 426 may comprise the majority of the interior volume. In some examples, the first lumen 426 and the inflation lumen 430 can be defined by an inner partition, e.g., a wall, within the catheter 410. In yet additional examples, the inflation lumen 430 can be integrated into a wall of the first lumen 426. The catheter 410 can be coupled with additional or alternative devices in other examples, such as a pressurization device configured to monitor the internal pressure of the coronary sinus, or a contrast agent injection device. In some examples, the catheter 410 can include an anchoring, positioning, and/or stabilizing component configured to maintain the position of the catheter necessary to occlude blood flow from the coronary sinus 402 to the right atrium 404. In some examples, the catheter 410 can include a non-inflatable, expandable member in lieu of the balloon 412. The balloon 412 is spherical in the example shown, but the balloon may also be conical, square, elongate, tapered, stepped, offset, rectangular, and/or combinations thereof.

In operation, contrast-heavy blood collected in the coronary sinus 402 flows into the distal portion 418 of the catheter 410 via distal port 414, which defines the distally terminal portion of the first lumen 426. The contrast-heavy blood is moved proximally through the first lumen 426, and depending on the configuration of the valve 421, travels from the coronary sinus 402 directly into the right atrium 404 or to the drainage device 423, e.g., a drainage bag. The valve 421 can be switched between a first, open configuration that allows blood to flow into the right atrium 404, and a second, closed configuration that prevents blood from entering the right atrium 404. In the closed configuration, contrast-heavy blood bypasses the right atrium 404 and continues to flow through the first lumen 426 to the drainage device 423, where the contrast-heavy blood can be deposited and discarded. To replace the discarded blood and maintain an approximately constant fluid level within the patient, saline can be administered to the patient. The valve 421 can be kept in the open configuration when no contrast agent remains in the patient, thereby allowing the contrast-free or contrast-reduced blood to flow freely into the right atrium via the first lumen 426. In various embodiments, the balloon catheter 410 may be positioned such that the valve 421 is positioned to funnel contrast-free or contrast-reduced blood directly into the right atrium 404 when in the open configuration, such that no blood is deposited into the inferior or superior vena cava.

The second, inflation lumen 430 connects the inflation device 424 to the balloon 412, such that inflation device 424 can inflate the balloon prior to injection of the contrast agent. The balloon 412 can remain inflated, under the control of the inflation device 424, such that the valve 421 comprises the only access point into the right atrium 404 during and/or after a PCI procedure.

Various contrast agents may also be used with the catheter 410. For example, contrast agents may be radiopaque and can be water soluble or water insoluble. Particular contrast agents employed may include metrizamide, iopamidol, iothalamate sodium, iodamide sodium, meglumine, and metals and metal oxides such as titanium, gold, silver, stainless steel, oxides thereof, aluminum oxide, and/or zirconium oxide.

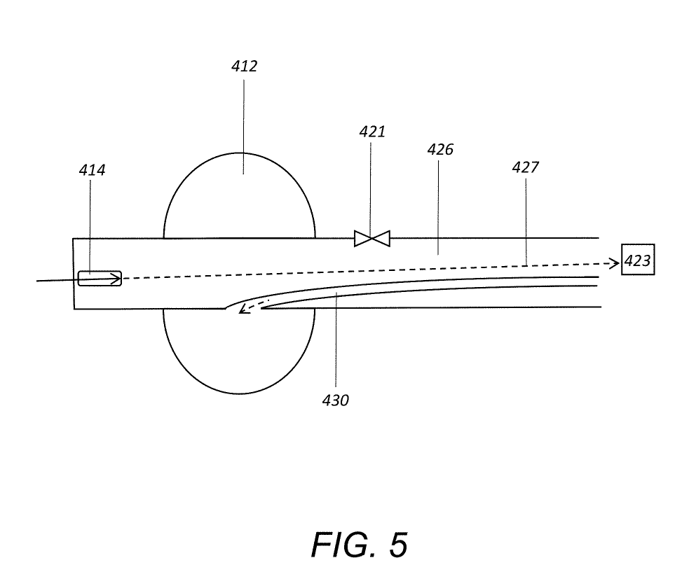

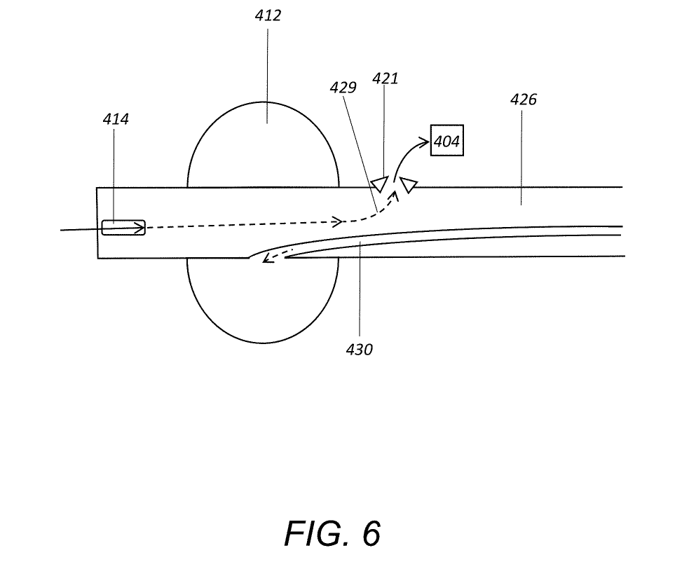

FIG. 5 shows a magnified schematic view of a portion of the balloon catheter 410 shown in FIG. 4, arranged in an opposite orientation, such that the distal port 414 is shown on the left. As shown, contrast-heavy blood 427 enters the distal port 414, bypasses the closed valve 421, and flows proximally through the first lumen 426 toward a drainage device 423. FIG. 6 shows a magnified schematic view of a portion of the balloon catheter 410 shown in FIG. 5. As shown, contrast-free blood 429 enters the first lumen 426 via the distal port 414. The open valve 421 allows the contrast-free blood to exit the first lumen 426 and flow into the right atrium 404. Means for controlling the configuration of the valve 421 may vary. In an embodiment, the valve 421 may be communicatively coupled to an external control device, e.g., a computer, configured to toggle the valve between open and closed configurations. The control device may be responsive to user input or may automatically alternate the configuration of the valve 421 in response to contrast agent administration, such that administration of the contrast agent triggers automatic closing of the valve 421. In other examples, the valve 421 can be physically coupled to a switch configured to control the valve configuration. In yet additional examples, the valve 421 may be responsive to one or more sensing devices coupled with the catheter 410 and configured to determine whether a contrast agent resides within the coronary sinus.

### EXAMPLES

The above Detailed Description is intended to be illustrative and not restrictive. The above-described embodiments (or one or more features or components thereof) can be used in varying combinations with each other unless clearly stated to the contrary. For example, the balloon catheter of FIG. 4 can be modified to include an additional lumen, like the balloon catheter of FIG. 2, such that contrast-heavy blood, which is moved proximally from the coronary sinus, can be filtered by a filtration device and returned to the right atrium in lieu of such blood being collected and discarded. Other embodiments can be used, such as by one of ordinary skill in the art upon reviewing the above Detailed Description. Also, various features or components have been grouped together to streamline the disclosure. This should not be interpreted as intending that an unclaimed disclosed feature is essential to any claim. Rather, inventive subject matter can lie in less than all features of a particular disclosed embodiment. Thus, the following claim examples are hereby incorporated into the Detailed Description, with each example standing on its own as a separate embodiment.

In Example 1, a method for preventing contrast-associated nephropathy may involve inserting a catheter into a patient's coronary sinus and blocking blood flow from the coronary sinus into the patient's right atrium. The blood may contain a contrast agent. The method may further involve draining the blood from the coronary sinus through a first lumen defined by the catheter while the blood flow is blocked, filtering the contrast agent from the blood which passes through the catheter, and returning contrast-free or contrast-reduced blood to the right atrium through a second lumen defined by the catheter. The second lumen can terminate in a port proximal to the coronary sinus.

In Example 2, the method of Example 1 can optionally be configured such that blocking blood flow comprises inflating a balloon coupled with the catheter and positioned between the coronary sinus and the right atrium.

In Example 3, the method of Example 2 can optionally be configured such that the catheter further comprises an inflation lumen coupled with an external inflation device and the balloon, the inflation lumen configured to direct an inflation substance into the balloon in response to activation of the inflation device.

In Example 4, the method of any one or any combination of Examples 1-3 can optionally be configured such that the catheter defines a distal port configured to receive blood from the coronary sinus and direct the blood into the first lumen.

In Example 5, a method for preventing contrast-associated nephropathy comprises inserting a catheter into a patient's coronary sinus, blocking blood from flowing into the patient's right atrium from the coronary sinus, and selectively draining blood from the coronary sinus into the right atrium through a first lumen defined by the catheter while the blood flow is blocked.

In Example 6, the method of Example 5 can optionally be configured such that selectively draining the blood comprises switching the catheter between an open configuration and a closed configuration by opening and closing a valve defined by the catheter.

In Example 7, the method of Example 6 can optionally be configured to further comprise maintaining the valve in the closed configuration when the blood contains a contrast agent, and depositing the blood into a drainage device coupled with the first lumen.

In Example 8, the method of Example 7 can optionally be configured to further comprise administering an amount of saline into the patient's blood stream.

In Example 9, the method of Example 6 can optionally be configured to further comprise maintaining the valve in the open configuration when the blood does not contain a contrast agent, and releasing the blood directly into the right atrium.

In Example 10, the method of any one or any combination of Examples 5-9 can optionally be configured such that blocking blood from flowing into the patient's right atrium comprises inflating a balloon coupled with the catheter and positioned between the coronary sinus and the right atrium.

In Example 11, the method of Example 10 can optionally be configured such that the balloon is never deflated.

In Example 12, the method of Example 10 can optionally be configured such that the catheter further comprises an inflation lumen coupled with an external inflation device and the balloon, the inflation lumen configured to direct an inflation substance into the balloon in response to activation of the inflation device.

In Example 13, the method of any one or any combination of Examples 5-12 can optionally be configured such that the catheter defines a distal port configured to receive blood from the coronary sinus.

In Example 14, the method of Example 6 can optionally be configured such that the valve is coupled with a control device configured to switch the valve between the open and closed configurations in response to administration of the contrast agent.

In Example 15, a balloon catheter comprises an elongate tube member defining a first lumen and configured for insertion into a patient's coronary sinus and a distal port defined by the elongate tube member, where the distal port is configured to receive blood from the coronary sinus. The catheter further comprises an inflatable balloon configured to block the blood from flowing into the patient's right atrium from the coronary sinus when inflated, and a valve positioned proximal to the balloon, where the valve is configured to switch between an open configuration and a closed configuration.

In Example 16, the balloon catheter of Example 15 can optionally be configured such that in the open configuration, the valve is configured to release the blood directly into the right atrium.

In Example 17, the balloon catheter of any one or any combination of Examples 15-16 can optionally be configured such that in the closed configuration, the valve is configured to deposit the blood into a drainage device coupled with a proximal end of the first lumen.

In Example 18, the balloon catheter of any one or any combination of Examples 15-17 can optionally be configured to further comprise an inflation lumen coupled with an external inflation device and the balloon, the inflation lumen configured to direct an inflation substance into the balloon in response to activation of the inflation device.

In Example 19, the balloon catheter of any one or any combination of Examples 15-18 can optionally be configured such that the elongate tube member includes a longitudinal preformed curve positioned proximal to the inflatable balloon.

In Example 20, the balloon catheter of Example 19 can optionally be configured such that the longitudinal preformed curve defines an arc of 30 degrees to 50 degrees, inclusive, over a length of 7.0 cm to 11.0 cm, inclusive.

Closing Notes:

The above Detailed Description includes references to the accompanying drawings, which form a part of the Detailed Description. The Detailed Description should be read with reference to the drawings. The drawings show, by way of illustration, specific embodiments in which the present balloon catheters and related methods can be practiced. These embodiments are also referred to herein as “examples.”

Certain terms are used throughout this patent document to refer to particular features or components. As one skilled in the art will appreciate, different people may refer to the same feature or component by different names. This patent document does not intend to distinguish between components or features that differ in name but not in function. For the following defined terms, certain definitions shall be applied unless a different definition is given elsewhere in this patent document. The terms “a,” “an,” and “the” are used to include one or more than one, independent of any other instances or usages of “at least one” or “one or more.” The term “or” is used to refer to a nonexclusive or, such that “A or B” includes “A but not B,” “B but not A,” and “A and B.” All numeric values are assumed to be modified by the term “about,” whether or not explicitly indicated. The term “about” refers to a range of numbers that one of skill in the art considers equivalent to the recited value (i.e., having the same function or result). In many instances, the term “about” can include numbers that are rounded to the nearest significant figure. The recitation of numerical ranges by endpoints includes all numbers and sub-ranges within and bounding that range (e.g., 1 to 4 includes 1, 1.5, 1.75, 2, 2.3, 2.6, 2.9, etc. and 1 to 1.5, 1 to 2, 1 to 3, 2 to 3.5, 2 to 4, 3 to 4, etc.). The terms “patient” and “subject” are intended to include mammals, such as for human or veterinary applications. The terms “distal” and “proximal” are used to refer to a position or direction relative to an operating physician. “Distal” and “distally” refer to a position that is distant from, or in a direction away from, the physician. “Proximal” and “proximally” refer to a position that is near, or in a direction toward, the physician.

The scope of the present balloon catheters and methods should be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended; that is, a device or method that includes features or components in addition to those listed after such a term in a claim are still deemed to fall within the scope of that claim. Moreover, in the following claims, the terms “first,” “second” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

What is claimed is:

1. A method for preventing contrast-associated nephropathy, comprising:
inserting a catheter into a patient's coronary sinus;
blocking blood flow from the coronary sinus into the patient's right atrium, wherein the blood contains a contrast agent;
draining blood from the coronary sinus through a first lumen defined by the catheter while the blood flow is blocked;
filtering the contrast agent from the blood which passes through the catheter; and
returning contrast-free or contrast-reduced blood to the right atrium through a second lumen defined by the catheter, wherein the second lumen terminates in a port proximal to the coronary sinus.

2. The method of claim 1, wherein blocking blood flow comprises inflating a balloon coupled with the catheter and positioned between the coronary sinus and the right atrium.

3. The method of claim 2, wherein the catheter further comprises an inflation lumen coupled with an external inflation device and the balloon, the inflation lumen configured to direct an inflation substance into the balloon in response to activation of the inflation device.

4. The method of claim 1, wherein the catheter defines a distal port configured to receive blood from the coronary sinus and direct the blood into the first lumen.

5. A method for preventing contrast-associated nephropathy, comprising:
inserting a catheter into a patient's coronary sinus;
blocking blood from flowing into the patient's right atrium from the coronary sinus; and
selectively draining blood from the coronary sinus into the right atrium through a first lumen defined by the catheter while the blood flow is blocked.

6. The method of claim 5, wherein selectively draining the blood comprises switching the catheter between an open configuration and a closed configuration by opening and closing a valve defined by the catheter.

7. The method of claim 6, further comprising:
maintaining the valve in the closed configuration when the blood contains a contrast agent; and
depositing the blood into a drainage device coupled with the first lumen.

8. The method of claim 7, further comprising administering an amount of saline into the patient's blood stream.

9. The method of claim 6, further comprising:
maintaining the valve in the open configuration when the blood does not contain a contrast agent; and
releasing the blood directly into the right atrium.

10. The method of claim 6, wherein the valve is coupled with a control device configured to switch the valve between the open and closed configurations in response to administration of the contrast agent.

11. The method of claim 5, wherein blocking blood from flowing into the patient's right atrium comprises inflating a balloon coupled with the catheter and positioned between the coronary sinus and the right atrium.

12. The method of claim 11, wherein the balloon is never deflated.

13. The method of claim 11, wherein the catheter further comprises an inflation lumen coupled with an external inflation device and the balloon, the inflation lumen configured to direct an inflation substance into the balloon in response to activation of the inflation device.

14. The method of claim 5, wherein the catheter defines a distal port configured to receive blood from the coronary sinus.

15. A balloon catheter comprising:
an elongate tube member defining a first lumen and configured for insertion into a patient's coronary sinus;
a distal port defined by the elongate tube member, the distal port configured to receive blood from the coronary sinus;
an inflatable balloon configured to block the blood from flowing into the patient's right atrium from the coronary sinus when inflated; and
a valve positioned proximal to the balloon, the valve configured to switch between an open configuration and a closed configuration.

16. The balloon catheter of claim 15, wherein, in the open configuration, the valve is configured to release the blood directly into the right atrium.

17. The balloon catheter of claim 15, wherein, in the closed configuration, the valve is configured to deposit the blood into a drainage device coupled with a proximal end of the first lumen.

18. The balloon catheter of claim 15, further comprising an inflation lumen coupled with an external inflation device and the balloon, the inflation lumen configured to direct an inflation substance into the balloon in response to activation of the inflation device.

19. The balloon catheter of claim 15, wherein the elongate tube member includes a longitudinal preformed curve positioned proximal to the inflatable balloon.

20. The balloon catheter of claim 19, wherein the longitudinal preformed curve defines an arc of 30 degrees to 50 degrees, inclusive, over a length of 7.0 cm to 11.0 cm, inclusive.
